(19) United States
(12) Patent Application Publication
(10) Pub. No.: US 2024/0130794 A1
(43) Pub. Date: Apr. 25, 2024

(54) AUTOMATIC OSS BASED FEATURE DETECTION AND DEVICE CHARACTERIZATION

(71) Applicant: KONINKLIJKE PHILIPS N.V., EINDHOVEN (NL)

(72) Inventors: Torre Michelle BYDLON, MELROSE, MA (US); Molly Lara FLEXMAN, MELROSE, MA (US)

(21) Appl. No.: 17/768,259

(22) PCT Filed: Oct. 15, 2020
(86) PCT No.: PCT/EP2020/079291

§ 371 Date: Apr. 11, 2022

Related U.S. Application Data
(60) Provisional application No. 62/916,342, filed on Oct. 17, 2019.

(51) Int. Cl. A61B 34/20; A61B 34/10; A61B 90/00

(52) U.S. Cl. CPC A61B 34/20; A61B 34/10; A61B 90/37; A61B 2034/102; A61B 2034/2051; A61B 2034/2061; A61B 2090/376; A61B 2090/378

(57) ABSTRACT

Various embodiments of the present disclosure encompass an optical shape sensing registration system employing an optical shape sensing guidewire (30) translatable within an over-the-wire device (40), and further employing an optical shape sensing registration controller (20) for controlling an autonomous device registration of an over-the-wire device (40). In operation, the optical shape sensing registration controller (20) automatically detects one or more sensing features of the optical shape sensing guidewire (30) (e.g., shape, curvature, temperature, vibration, strain, etc.) from an optical shape sensing of a translation of the optical shape sensing guidewire (30) within the over-the-wire device (40), and then automatically determines one or more registration characteristics of the over-the-wire device (40) (e.g., a device type, a length, a diameter, a color, a hub type, treatment device(s), anatomical image(s), anatomical model(s), anatomical location(s), procedure type etc.) from an automatic detection of the sensing feature(s) of the optical shape sensing guidewire (30).

### FIELD OF THE INVENTION

The present disclosure generally relates to optical shape sensing. The present disclosure specifically relates to optical shape sensing as a basis for automatically detecting feature(s) of an optical shape sensing guidewire and automatically determining characteristic(s) of an over-the-wire device having the optical shape sensing guidewire translated therein.

### BACKGROUND OF THE INVENTION

Optical shape sensing (OSS) uses light along a multicore optical fiber for device localization and navigation during surgical interventions. The principle involved utilizes distributed strain measurements in the optical fiber defined by characteristic Rayleigh backscatter or controlled grating patterns (e.g., Fiber Bragg Gratings). The shape along the optical fiber begins at a specific point along the sensor, known as the launch or z=0, and the subsequent shape position and orientation are relative to that point.

Optical shape sensing fibers can be integrated into medical devices in order to provide live guidance of the devices during minimally invasive procedures. The integrated fiber provides the position and orientation of the entire device, such as, for example, a catheter loaded onto a shape-sensed guidewire for navigation to an anatomical target whereby the shape-sensed guidewire facilitates an overlay of the catheter in a pre-operative computed tomography image of the anatomical target.

Registration characteristics of the medical device must be known to support spatial tracking of the medical device during a minimally invasive procedure. Such registration characteristics of the medical device need to be determined in a manner that minimizes any interruption to the workflow of the minimally invasive procedure.

### SUMMARY OF THE INVENTION

For numerous and various applications involving a spatial tracking of an over-the-wire (OTW) device, the present disclosure describes controllers, systems and methods for automatically determining required device characteristics needed for an accurate, robust spatial tracking of the OTW device via an optical shape sensing (OSS) guidewire in a manner that minimizes any interruption to the workflow of the application; or for annotation, reporting, documentation, or the like. Examples of such applications include, but are not limited to, vascular applications (e.g., via catheters, sheaths, deployment systems, etc.), endoluminal applications (e.g., via endoscopes) and orthopedic applications (via k-wires & screwdrivers).

The present disclosure may be embodied as:
(1) an OSS registration controller of the present disclosure;
(2) an OSS registration system incorporating an OSS registration controller of the present disclosure; and
(3) an OSS registration method utilizing an OSS registration controller of the present disclosure.

In various embodiments an OSS registration system of the present disclosure encompasses an OSS guidewire translatable within an OTW device, and further encompasses an OSS registration controller for controlling an autonomous device registration of the OTW device.

In operation, the OSS registration controller (1) automatically detects one or more sensing features of the OSS guidewire (e.g., shape, curvature, temperature, vibration, strain, twist, alpha, etc.) from an optical shape sensing of a translation of the OSS guidewire within the over-the-wire device, and (2) automatically determines one or more registration characteristics of the OTW device (e.g., a type, a length, a diameter, a color, a hub, treatment device(s), anatomical image(s), anatomical model(s), anatomical location(s), etc.) from an automatic detection of the sensing feature(s) of the OSS guidewire.

Various embodiments of an OSS registration controller of the present disclosure encompass a non-transitory machine-readable storage medium encoded with instructions for execution by one or more processors for controlling an autonomous device registration of an OTW device translatable within a OSS guidewire.

The non-transitory machine-readable storage medium includes the instructions to (1) automatically detect one or more sensing features of the OSS guidewire (e.g., shape, curvature, temperature, vibration, strain, etc.) from an optical shape sensing of a translation of the OSS guidewire within the OTW device, and (2) automatically determine one or more registration characteristics of the OTW device (e.g., a type, a length, a diameter, a color, a hub, treatment device(s), anatomical image(s), anatomical model(s), anatomical location(s), etc.) from an automatic detection of the sensing feature(s) of the OSS guidewire.

Various embodiments of an OSS registration method of the present disclosure utilizing an OSS registration controller for controlling an autonomous device registration of an OTW device translatable within a OSS guidewire.

The OSS registration method involves the optical shape sensing registration characteristic controller (1) automatically detecting one or more sensing features of the OSS guidewire (e.g., shape, curvature, temperature, vibration, strain, twist, alpha, etc.) from an optical shape sensing of a translation of the OSS guidewire within the OTW device, and (2) automatically determining one or more registration characteristics of the OTW device (e.g., a device type, a length, a diameter, a color, a hub type, treatment device(s), anatomical image(s), anatomical model(s), anatomical location(s), etc.) from an automatic detection of the sensing feature(s) of the OSS guidewire.

The foregoing embodiments and other embodiments of the present disclosure as well as various structures and advantages of the present disclosure will become further apparent from the following detailed description of various embodiments of the present disclosure read in conjunction with the accompanying drawings. The detailed description and drawings are merely illustrative of the present disclosure rather than limiting, the scope of the present disclosure being defined by the appended claims and equivalents thereof.

### BRIEF DESCRIPTION OF THE DRAWINGS

The present disclosure will present in detail the following description of exemplary embodiments with reference to the following figures wherein:

FIG. 1 illustrates an exemplary embodiment of an OSS registration system in accordance with the present disclosure;

FIG. 2 illustrates an exemplary embodiment of a OSS registration method in accordance with the present disclosure;

FIG. 3 illustrates flowcharts representative of a first exemplary embodiment of the OSS registration method of FIG. 2 in accordance with the present disclosure;

FIG. 4 illustrates a flowchart representative of an exemplary embodiment of an automatic hub detection method in accordance with the present disclosure;

FIGS. 5A and 5B illustrate exemplary graphical automatic hub detections in accordance with the present disclosure;

FIG. 6 illustrates a flowchart representative of an exemplary embodiment of an automatic OTW device measurement method in accordance with the present disclosure;

FIG. 7 illustrates a flowchart representative of an exemplary embodiment of an OSS peak measurement method in accordance with the present disclosure;

FIG. 8 illustrates an exemplary OSS peak measurement in accordance with the present disclosure;

FIG. 9 illustrates a flowchart representative of an exemplary embodiment of an OSS distal curvature measurement method in accordance with the present disclosure;

FIG. 10 illustrates an exemplary OSS distal curvature measurement in accordance with the present disclosure;

FIG. 11 illustrates a flowchart representative of an exemplary embodiment of an OSS shape matching measurement method in accordance with the present disclosure;

FIG. 12 illustrates an exemplary OSS shape matching measurement in accordance with the present disclosure;

FIG. 13 illustrates flowcharts representative of a second exemplary embodiment of the OSS registration method of FIG. 2 in accordance with the present disclosure; and

FIG. 14 illustrates an exemplary embodiment of a OSS registration controller in accordance with the present disclosure.

### DETAILED DESCRIPTION OF THE PREFERRED EMBODIMENTS

The present disclosure improves upon a device registration during an application involving a spatial tracking of an over-the-wire (OTW) device by (1) an automatic detection one or more sensing features of an optical shape sensing (OSS) guidewire (e.g., shape, curvature, temperature, vibration, strain, twist, alpha, etc.) from an optical shape sensing of a translation of the OSS guidewire within the over-the-wire device, and (2) an automatic determination of one or more registration characteristics of the OTW device (e.g., a type, a length, a diameter, a color, a hub, treatment device(s), anatomical image(s), anatomical model(s), anatomical location(s), etc.) from an automatic detection of the sensing feature(s) of the OSS guidewire.

For purposes of the description and claims of the present disclosure:
(1) terms of the art including, but not limited to, “optical shape sensing (OSS)”, “OSS guidewire”, “over-the-wire (OTW) device”, “hub”, “hub template”, “autonomous (and tenses thereof)”, “automatic (and tenses thereof)” are to be interpreted as known in the art of the present disclosure and as exemplary described in the present disclosure;
(2) more particularly, the term “OSS guidewire” broadly encompasses a wire, spring or the like, as known in the art of the present disclosure and hereinafter conceived, incorporating optical shape sensing for guiding a spatial positioning of an OTW device;
(3) examples of an OTW device include, but are not limited to, a catheter, a deployment system and a sheath;
(4) more particularly, the term “hub” broadly encompasses any object, as known in the art of the present disclosure and hereinafter conceived, for referencing an optical shape sensing of a OTW device via an OSS guidewire. Examples of a hub include, but are not limited to, a unicath hub, a luer lock hub, an over-catheter hub, a hemostatic valve hub, a guidewire torque hub, an introducer hub.
(5) more particularly, the term “hub template” broadly encompasses any shape profile, curvature profile or strain profile of an OSS guidewire formed within a hub;
(6) the term “autonomous device registration” broadly encompasses autonomous action or actions performed by a controller that is(are) for purposes of determining registration characteristic(s) of an OTW device relevant to a spatial tracking of the OTW device as exemplary described in the present disclosure;
(7) the term “automatic” broadly encompasses autonomous action or actions performed by a controller that is(are) dependent upon optical shape sensing of a translation of an OSS guidewire within an OTW device as exemplary described in the present disclosure;
(8) the term “sensing feature” broadly encompasses a feature of an OSS guidewire derived from an interrogation of the OSS guidewire as known in the art of the present disclosure. The sensing feature may be derived from the OSS guidewire itself, from a hub that induces a feature in the OSS guidewire, or an OTW device that induces a feature in the OSS guidewire. Examples of sensing features of an OSS guidewire include, but are not limited to, a shape, a curvature, a temperature, a vibration and a strain of a segment or an entirety of the OSS guidewire;
(9) the term “registration characteristic” broadly encompasses a characteristic of an OTW device serving as a variable or a registration characteristic of a spatial registration of the OTW device with an OSS device for tracking purposes as known in the art of the present disclosure. Examples of registration characteristics of an OTW device include, but are not limited to, a device type, a length, a diameter and a color of the OTW device, and a hub type, treatment device(s), anatomical image(s), anatomical model(s), anatomical location(s), and procedure type associated with an OTW device;
(10) the term “look-up-table” or “LUT” encompasses a database of templates and device characteristics that may be defined prior to the procedure or during the procedure. Examples of such databases include, but are not limited to, look-up-tables as known in art of the present disclosure, and artificial intelligence generated databases.
(11) the term “controller” broadly encompasses all structural configurations, as understood in the art of the present disclosure and as exemplary described in the present disclosure, having main circuit board(s) and/or integrated circuit(s) for controlling an application of various principles of the present disclosure as exemplary described in the present disclosure;
(12) the term “application module” broadly encompasses an application incorporated within or accessible by controller consisting of an electronic circuit (e.g., electronic components and/or hardware) and/or an executable program (e.g., executable software stored on non-transitory computer readable medium(s) and/or firmware) for executing a specific application of the present disclosure as exemplary described in the present disclosure; and
(13) the terms “signal” and “data”” broadly encompasses all forms of a detectable physical quantity or impulse (e.g., voltage, current, or magnetic field strength) as understood in the art of the present disclosure and as exemplary described in the present disclosure for transmitting information and/or instructions in support of applying various inventive principles of the present disclosure as subsequently described in the present disclosure. Signal/data communication various components of the present disclosure may involve any communication method as known in the art of the present disclosure including, but not limited to, signal/data transmission/reception over any type of wired or wireless datalink and a reading of signals/data uploaded to a computer-usable/computer readable storage medium.

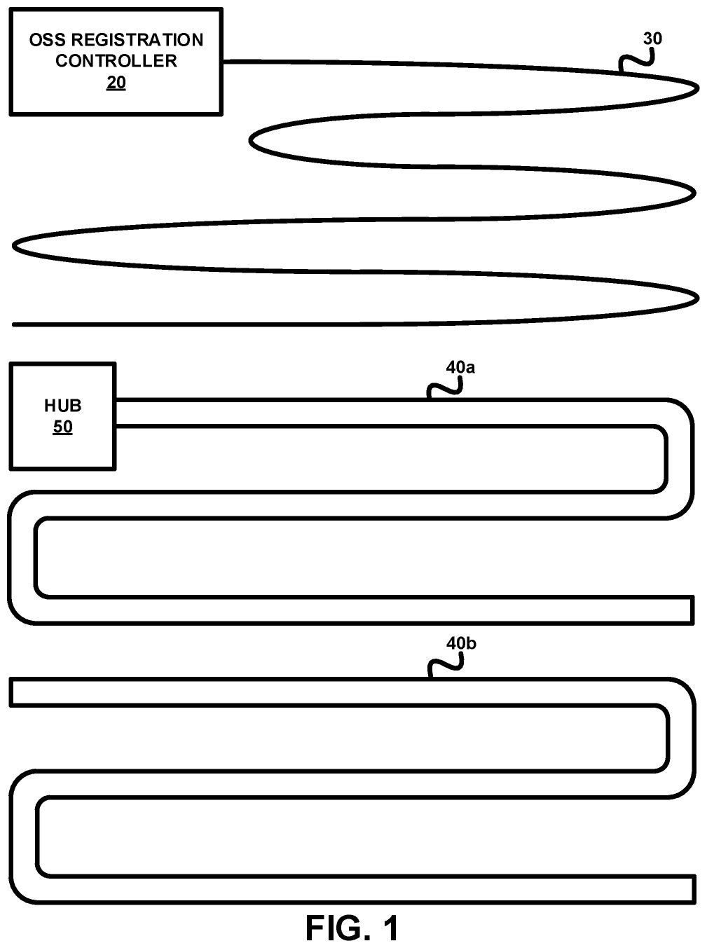

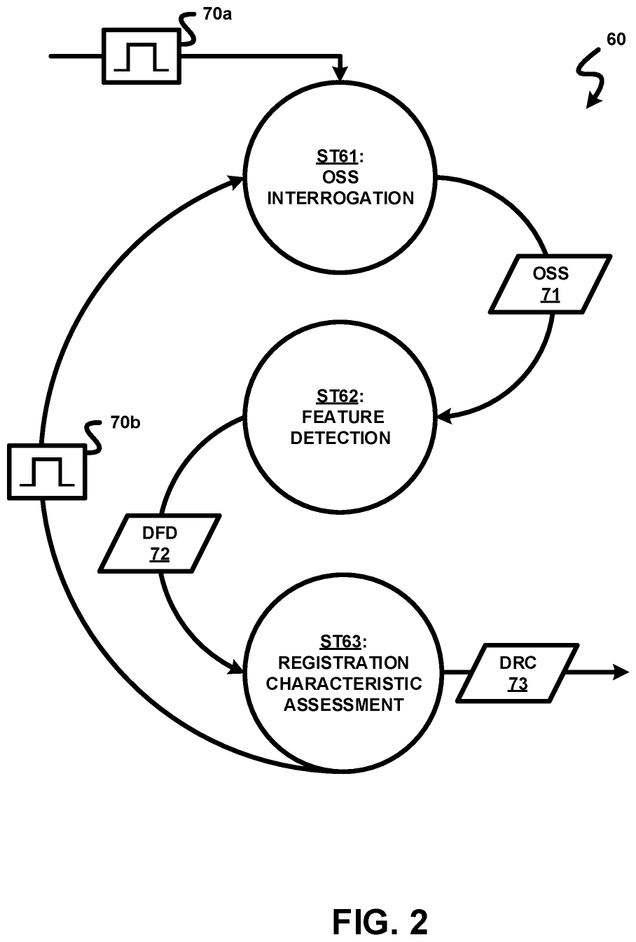

To facilitate an understanding of the present disclosure, the following description of FIGS. 1 and 2 teaches an exemplary embodiments of an OSS registration system and an OSS registration method, respectively, in accordance with the present disclosure. From the description of FIGS. 1 and 2, those having ordinary skill in the art of the present disclosure will appreciate how to apply the present disclosure to make and use additional embodiments of an OSS registration system and an OSS registration method of the present disclosure for any type of OTW device.

Referring to FIG. 1, an OSS registration system of the present disclosure employs an OSS registration controller 20 and an OSS guidewire 30.

In practice, OSS guidewire 30 is a guidewire having an optical shape sensor with fiber optics embedded therein as known in the art of the present disclosure.

In one exemplary embodiment, the optical shape sensor is based on fiber optic Bragg grating sensors. A fiber optic Bragg grating (FBG) is a short segment of optical fiber that reflects particular wavelengths of light and transmits all others. This is achieved by adding a periodic variation of the refractive index in the fiber core, which generates a wavelength-specific dielectric minor. A fiber Bragg grating can therefore be used as an inline optical filter to block certain wavelengths, or as a wavelength-specific reflector.

More particular, a FBG sensor may use Fresnel reflection at each of the interfaces where the refractive index is changing. For some wavelengths, the reflected light of the various periods is in phase so that constructive interference exists for reflection and, consequently, destructive interference for transmission. The Bragg wavelength is sensitive to strain as well as to temperature. This means that Bragg gratings can be used as sensing elements in fiber optic sensors. In an FBG sensor, the measurand (e.g., strain) causes a shift in the Bragg wavelength.

In a second exemplary embodiment, the optical shape sensor is based on inherent backscatter. One such approach uses Rayleigh scatter (or other scattering) in standard single-mode communications fiber. Rayleigh scatter occurs as a result of random fluctuations of the index of refraction in the fiber core. These random fluctuations can be modeled as a Bragg grating with a random variation of amplitude and phase along the grating length. By using this effect in three or more cores running within a single length of multi-core fiber, the 3D shape and dynamics of the surface of interest can be followed.

One advantage of OSS guidewire 30 is that various sensor elements can be distributed over the length of a fiber. Incorporating three or more cores with various sensors (gauges) along the length of a fiber that is embedded in a structure permits a three-dimensional form of such a structure to be precisely determined, typically with better than 1 mm accuracy. Along the length of the fiber, at various positions, a multitude of FBG sensors can be located. From the strain measurement of each FBG, the curvature of the structure can be inferred at that position. From the multitude of measured positions, the total three-dimensional form may be determined.

Still referring to FIG. 1, in practice, OSS registration controller 20 or another controller interfaces with an optical interrogator including or working with an optical source as known in the art of the present disclosure whereby controller 20 (or the other controller) controls an operation of an optical interrogator for sending and receiving optical signals from OSS guidewire 30 representative of sensing feature(s) of OSS guidewire 30 as known in the art of the present disclosure (e.g., a shape, a curvature, a temperature, a vibration and a strain of a segment or an entirety of the OSS guidewire (30)). Controller 20 (or the other controller) further controls a reconstruction of the shape of OSS guidewire 30 based on the received optical signals (i.e., shape sensing data) as known in the art of the present disclosure.

Still referring to FIG. 1, the OSS registration system of the present disclosure further employs OTW devices having in the form of a catheter 40a having a hub 50 attached to a proximal end thereof (e.g., via a Luer lock) and a catheter 40b, which may be equipped with a hub 50 (not shown) attachable to a proximal end thereof. Hub 50 includes a distinct template formed in a hub body as known in the art of the present disclosure to distinguish a portion of OSS guidewire 30 within the hub body via the shape sensing data.

In practice, OTW devices may be in alternative forms as known in the art of the present disclosure including, but not limited to, a deployment system and a sheath.

Also in practice, catheters 40 may be front loaded or back loaded, manually or robotically onto OSS guidewire 30 whereby OSS registration controller 20 controls a determination a device registration of catheters 40 involving an autonomous action or actions performed by OSS registration controller 20 that is(are) for purposes of determining registration characteristic(s) of catheters 40 relevant to a spatial tracking of catheters 40.

Examples of registration characteristics of catheters 40 include a catheter type, a length, a diameter and a color of the catheters 40, and a hub type, treatment device(s), anatomical image(s), anatomical model(s), anatomical location(s), and procedure type(s) associated with catheters 40.

In one exemplary embodiment, a registration characteristic of catheters 40 is the type of hub 50 attached or attachable thereto. To determine this registration characteristic, OSS registration controller 20 employs a hub detection module for automatically detecting of a hub template of hub 50 relative to the OSS guidewire 30 from an optical shape sensing of a translation of the OSS guidewire 30 through the hub template as will be further described in the present disclosure or by automatically detecting a device type of catheter 40b from an optical shape sensing of a translation of the OSS guidewire 30 within the catheter 40b as will be further described in the present disclosure.

In a second exemplary embodiment, a registration characteristic of catheters 40 is a length of catheters 40. To determine this registration characteristic, OSS registration controller 20 employs a OTW device measurer for automatically deriving a measurement of a length of catheter 40 from an optical shape sensing of a translation of the OSS guidewire 30 through catheter 40 as will be further described in the present disclosure.

Referring to FIG. 2, a state machine 60 representative of an OSS registration method 60 of the present disclosure implemented as OSS guidewire (e.g., OSS guidewire 30 of FIG. 1) is translated within an OTW device (e.g., catheters 40 of FIG. 1).

A state ST61 of state machine 60 encompasses a OSS registration controller of the present disclosure (e.g., controller 20 of FIG. 1) controlling, via a command signal 70a, an interrogation of the OSS guidewire as translated within the OTW device.

A state ST62 of state machine 60 encompasses the OSS registration controller processing OSS data 71 generated via the interrogation of the OSS guidewire to detect one or more sensing feature(s) of the OSS guidewire as known in the art of the present disclosure (e.g., a shape, a curvature, a temperature, a vibration and a strain of a segment or an entirety of the OSS guidewire).

A state ST63 of state machine 60 encompasses the OSS registration controller processing detected feature data 72 indicative of the detected features of the OSS guidewire to assess if one or more registration characteristics of the OTW device may be derived from the detected features of the OSS guidewire.

If one or more registration characteristics of the OTW device are derived from the detected features of the OSS guidewire, then the OSS registration controller outputs device registration characteristic data 73 of the OTW device for purposes of facilitating a spatial tracking of the OTW device (e.g., a device type, a length, a diameter and a color of the OTW device, and a hub type, treatment device(s), anatomical image(s), anatomical model(s), anatomical location(s), and procedure type associated with an OTW device).

More particularly, the detected features of the OSS guidewire may be used to delineate an anatomical location. For example, a detected shape of an OSS guidewire may correspond to a particular shape of an anatomical location, whereby a detection of that shape of the OSS guidewire as the OSS guidewire is being navigated within an anatomical region specifies a positioning and/or an orientation of the OSS guidewire (and OTW device) at the anatomical location.

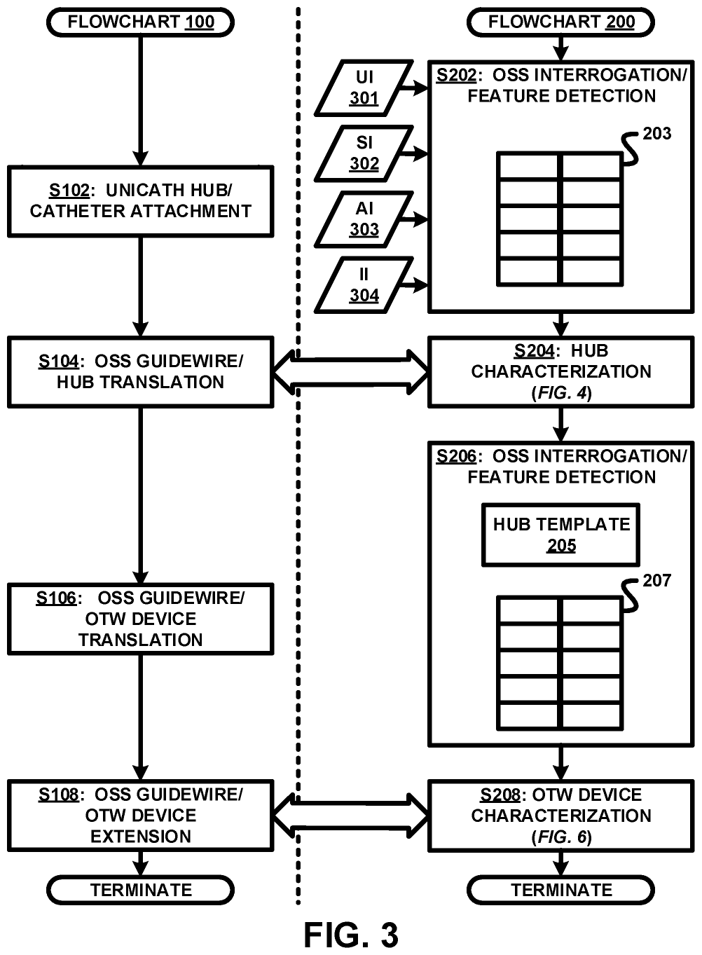

If one or more registration characteristics of the OTW device are incapable of being derived from the detected features of the OSS guidewire, then the OSS registration controller controlling, via a command signal 70b, an interrogation of the OSS guidewire as further translated within the OTW device whereby states ST61-ST63 are repeated until the registration characteristic(s) of the OTW device are derived from the detected features of the OSS guidewire. To facilitate a further understanding of the present disclosure, the following description of FIGS. 3-13 teaches exemplary embodiments of the OSS registration system of FIG. 1 and the OSS registration method of FIG. 2, respectively, in accordance with the present disclosure for detecting OSS guidewire feature(s) as the basis for capturing registration characteristic(s) of OTW device(s) as taught by FIG. 2. From the description of FIGS. 3-13, those having ordinary skill in the art of the present disclosure will appreciate how to apply the present disclosure to make and use additional embodiments of the OSS registration system of FIG. 1 and the OSS registration method of FIG. 2 for any type of OTW device.

FIG. 3 illustrates flowcharts 100 and 200 representative of an OSS registration method of the present disclosure that is particularly applicable to an OTW device within an attached hub, such as, for example, catheter 40 of FIG. 1.

Referring to FIG. 3, flowchart 100 and flowchart 200 encompass a workflow of user actions and controller executions for determining registration characteristics defining a spatial tracking of catheter 40 relative to attached hub 50 or another OTW device relative to an attached hub.

Generally, an implementation of a first phase of user actions/controller executions consist of a stage S102 and a stage S104 of flowchart 100 and a stage S202 and a stage S204 of flowchart 200 for detecting hub template as OSS guidewire 30 is translated through hub 50 to thereby determine a type of hub 50.

An implementation of a second phase of user actions/controller executions consist of a stage S106 and a stage S108 of flowchart 100 and a stage S206 and a stage S208 of flowchart 200 for measuring catheter 40 as OSS guidewire 30 is translated through catheter 40.

Prior to a commencement of flowcharts 100 and 200, a look-up-table (LUT) 203 of an array of pre-defined hub types and associated distinct templates is defined whereby a detected hub template enables a lookup of the corresponding hub type as will be further described in the present disclosure. In practice, the different hub templates may include unicath hubs with varying degrees of curvature or different shapes.

Additionally, a LUT 207 of an array of pre-defined catheters and associated shape or curvature profiles may be defined whereby a detected shape or curvature of a catheter enables a lookup of a particular type of catheter. For example, the shape or curvature profiles of several known devices (e.g., a cobra catheter, a SOS catheter, a VS1 catheter, etc.) may be included in LUT 207.

Still referring to FIG. 3, stage S102 of flowchart 100 encompasses a first user action involving an attachment of hub 50 to catheter 40, and stage S104 of flowchart 100 encompasses a second user action of manually or robotically translating OSS guidewire 30 through hub 50. Stages S102 and S104 of flowchart 100 initiate stage S202 and stage S204 of flowchart 200 for an automatic detection of which type of hub 50 is attached to catheter 40. The initiation of stage S202 and S204 of flowchart 200 occur from one or more of the following inputs.

In one exemplary embodiment, a user input 301 may provide an indication to controller 20 that hub 50 is attached to catheter 40 and an insertion of OSS guidewire 30 into hub 50 is about to start. User input 310 may be in the form of visual, verbal and/or manual cues.

In a second exemplary embodiment, a sensor may be incorporated into the hub 50 whereby the sensor sends a sensor input signal 302 to controller 20 when the sensor senses an attachment of the hub 50 to catheter 40 or the sensor senses OSS guidewire 30 is in contact with the hub 50. Further, additional sensors may be incorporated into other devices, at the table, in the room, etc. for sensing stage S102 and/or S104 have occurred.

In a third exemplary embodiment, controller 20 (or another controller) may execute an algorithm continuously looking at the shape data from the OSS guidewire 30 to detect a formation of a minimal curvature in a distal end of OSS guidewire 30. Once the algorithm sees the minimal curvature in the shape data, an algorithm signal 303 triggers stage S202.

In a fourth exemplary embodiment, imaging information 304 extracted from x-ray, ultrasound, optical/camera, etc. imaging may also be used to identify when catheter 40 is present, or if a hub 30 is in use. The identification is used as a trigger to initiate stage S202.

The first phase implemented by stage S202 and S204 involve a continuous processing of the shape sensing data of OSS guidewire 30 during stage S202 as the OSS guidewire is translated through hub template during stage S104 whereby, during stage S204, controller 20 attempts to match the current shape data of OSS guidewire 30 to the pre-defined hub templates in the LUT 203. The pre-defined hub template with the lowest error is defined as the hub with the best match and that hub type (or classification) is saved.

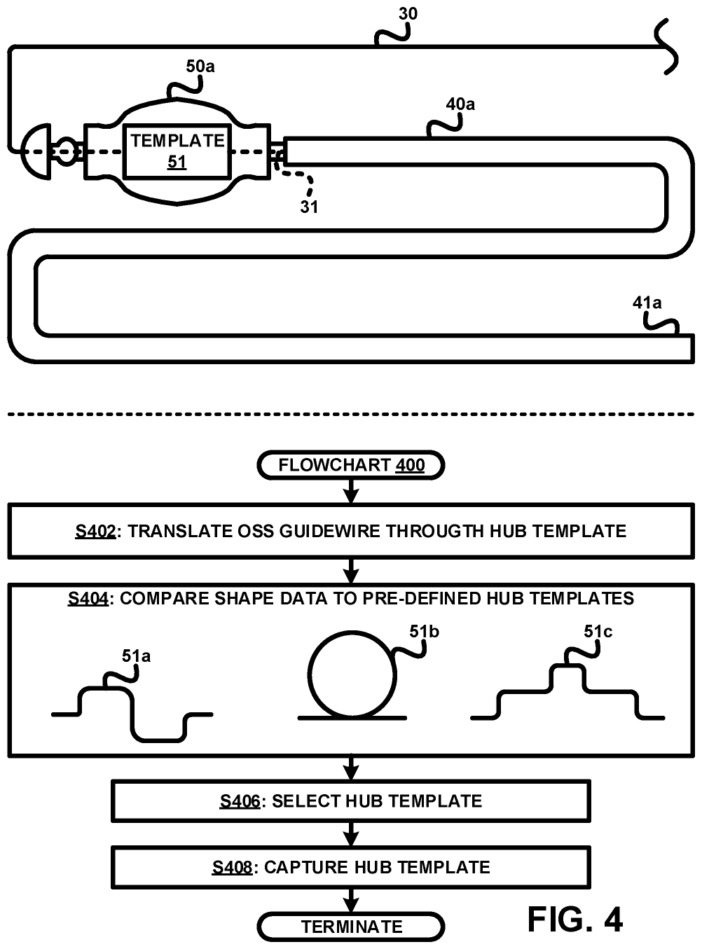

FIG. 4 illustrate an exemplary embodiment of the first phase.

Referring to FIG. 4, a stage S402 of a flowchart 400 encompasses an optical shape sensing of a translation of OSS guidewire 30 through hub template subsequent to an attachment of hub 50 to catheter 40 and front loading or a back loading of the hub 50 onto the OSS guidewire 30 as shown. During stage S402, a user may click a button to initialize controller 20 or alternatively, controller 20 may always be running and can detect when hub 50 is connected to catheter 40 based on signal processing and signal optimization techniques known in the art.

A stage S404 of flowchart 400 encompasses controller 20 comparing current shape data of OSS guidewire 30 to pre-defined hub templates of LUT 203, whereby the pre-defined hub template with the lowest error is selected as the detected hub template. The error function may be in accordance with the following equation [1].

$$Err = min ( \frac{{\sum }_{idx = 1}^{templat e . l ength} ( {shape}_{{idx}_{match} + idx} . kappa - {template}_{idx} . kappa )}{{\sum }_{idx = 1}^{template . length} ( {template}_{idx} . kappa )} ) for {idx}_{m a t c h} = 1 … shape . length \qquad [ 1 ]$$

In practice, the area under the hub template may be normalized, otherwise smaller templates may be favored over larger ones.

The template position may be shown to the user. If an incorrect template position or template has been selected, then the user can ‘window’ the search range. The search range can also be windowed without user input to exclude a portion of the OSS guidewire 30 that is inside or outside the body (by looking at the gradient in the axial strain), or a very distal portion of the sensor (last 10 cm for example).

Once the template position is selected during a stage S406 of flowchart 400, a stage S408 of flowchart 400 encompasses controller 20 capturing (extracting) the actual hub template curvature from the shape for the region that aligns with the stored hub template.

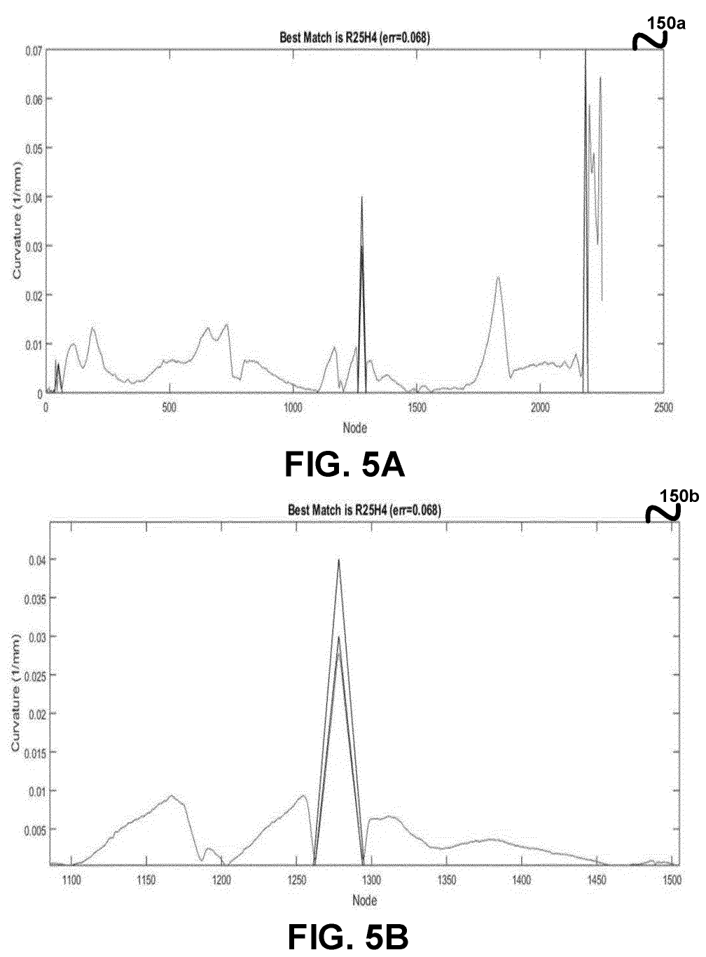

FIG. 5A shows pre-defined hub templates matched to an optically shaped sensed curvature of OSS guidewire 30 translating through hub template, and FIG. 5B shows a zoomed in view of the correct match.

Referring back to FIG. 3, stage S106 of flowchart 100 encompasses a third user action of manually or robotically translating OSS guidewire 30 through catheter 40, and stage S108 of flowchart 100 encompasses a fourth user action of manually or robotically extension of a tip of OSS guidewire 30 from hub 50. Stages S106 and S108 of flowchart 100 initiate stage S206 and stage S208 of flowchart 200 for an automatic measurement of a length of catheter 40.

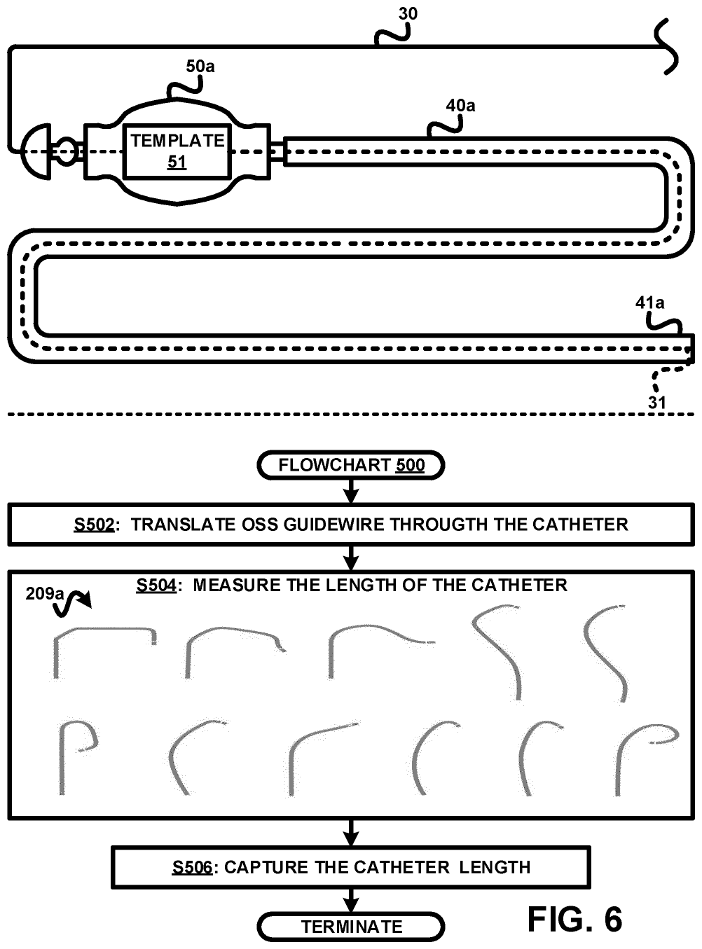

FIG. 6 illustrate an exemplary embodiment of the second phase.

Referring to FIG. 6, a stage S502 of a flowchart 500 encompasses an optical shape sensing of a manual translation or a robotic translation of OSS guidewire 30 through catheter 40 as shown subsequent the optical shape sensing of the manual translation or the robotic translation of OSS guidewire 30 through hub template as shown in FIG. 4.

A stage S504 of flowchart 500 encompasses detector 21 of controller 20 automatically deriving a measurement of a length of catheter 40 relative to hub 50 from an optical shape sensing of the stage S502 translation OSS guidewire 30 through catheter 40 involving an alignment or an extension of a distal tip 31 of OSS guidewire 30 with a distal tip 41 of catheter 40. To this end, detector 21 of controller 20 may include a database 209 of catheter shapes 51a including, but not limited to, a tiger catheter, a jacky catheter, an amplatz left catheter, a LCB catheter, a RCB catheter, a Judkins left catheter, a Judkins right catheter, a multipurpose A2 catheter, a IM catheter, a 3D lima catheter and an IM VB-1 catheter.

A stage S506 of flowchart encompasses controller 20 capturing the measured catheter length for optical shape sensing reconstruction of a shape of catheter 40 as known in the art of the present disclosure or hereinafter conceived.

Exemplary embodiments of stage S504 will now be described herein.

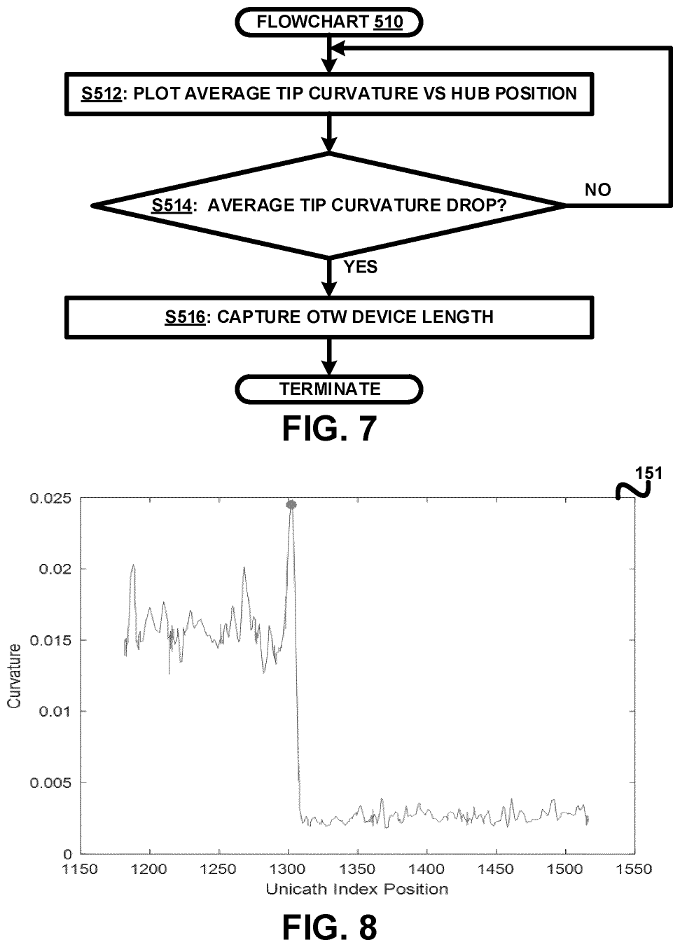

FIG. 7 illustrates a flowchart 510 represents a OSS peak measurement method of the present disclosure. Referring to FIG. 7, a stage S512 of flowchart 510 encompasses controller 20 saving an average curvature at tip 31 of OSS guidewire 30 at every time point while OSS guidewire 30 starts at the proximal end of catheter 40 and is pushed to the distal tip 41 of catheter 40 and then beyond, and plotting the average tip curvature versus the position of the hub 50. Stage S512 results in a large, rapid increase in the average curvature of tip 31 of OSS guidewire 30 when it reaches the distal tip 41 of catheter 40, followed by a steep decrease of the average curvature of tip 31 of OSS guidewire 30. The spike in the curvature defines the point at which the tips 31, 41 of the two devices 30, 40 are aligned.

For example, FIG. 8 illustrate a graph 151 of a plot of a large, rapid increase in the average curvature of tip 31 of OSS guidewire 30 when it reaches the distal tip 41 of catheter 40, followed by a steep decrease of the average curvature of tip 31 of OSS guidewire 30, whereby the spike in the curvature defines the point at which the tips 31, 51 of the two devices 30, 50 are aligned.

Referring back to FIG. 7, stage S514 of flowchart 510 encompasses controller 20 continually monitoring the plot for the average tip curvature drop whereby, upon controller 20 identifying the average tip curvature drop, a stage S516 of flowchart 510 encompasses controller 20 utilizing this spike in the average tip curvature extract the unicath hub index position may be extracted at the peak to thereby define the length of the catheter 40. From here additional device characteristics may be captured by matching the current OTW device or hub to the pre-defined look-up-table of device characteristics.

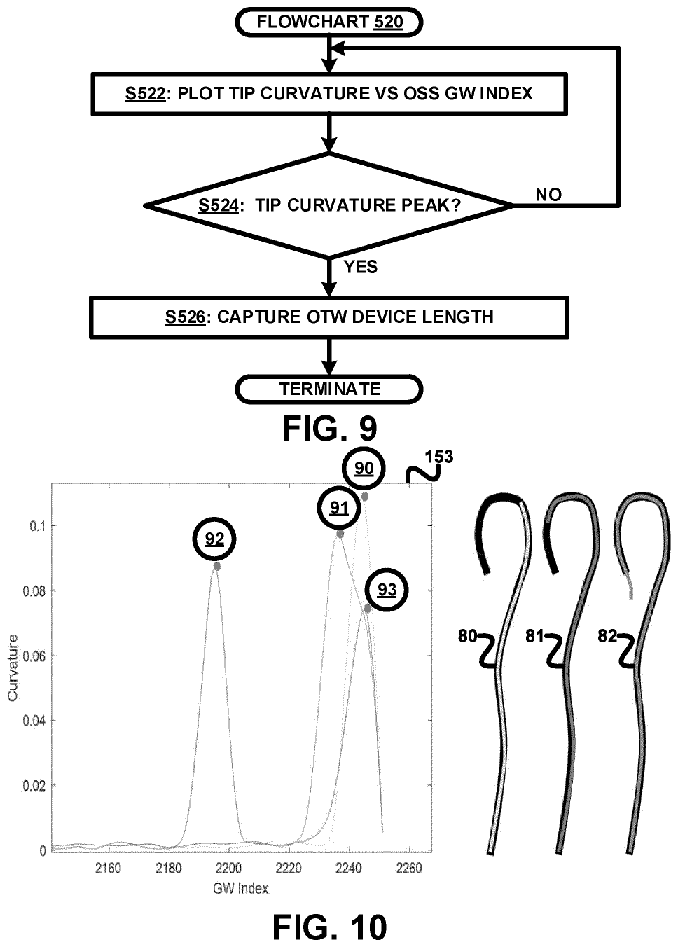

FIG. 9 illustrates a flowchart 520 represents a OSS distal curvature method of the present disclosure. Referring to FIG. 9, a stage S522 of flowchart 500 encompasses controller 20 saving a curvature at a distal tip of OSS guidewire 30 at every time point while OSS guidewire 30 starts at the proximal end of catheter 40 and is pushed to the distal tip 41 of catheter 40 and then beyond, and plotting the tip curvature versus the position of the hub 50. At each frame, peaks in the curvature signal are found. Each peak is given a label, such as A, B, C or 1, 2, 3. The label is initialized with the first frame of data. With each new frame, the label is applied to the peak closest to the last one—in this way a specific curve in the catheter 40 gets a label and that curve with respect to the OSS guidewire 30 can always be located.

FIG. 10 illustrates an exemplary graph 153 of a plotting of the curvature of the distal tip of OSS guidewire 30. The coloured lines are from three (3) different time points with the distal tip of OSS guidewire 30 at different positions 80, 81 and 82 within the catheter 40. The numbered circle indicates a label for each peak in the curvature profile.

More particularly, FIG. 10 shows a first time point when the OSS guidewire 30 is still within the catheter 40 (blue). At this time point a curve is identified in the OSS guidewire 30 and the curve is labeled (1). At a second time point (purple) when the OSS guidewire 30 is still within the catheter 40 but a little further in, curve (1) has shifted more proximally on the OSS guidewire 30. As this keeps going curve (1) ends up more and more proximal on the OSS guidewire 30. At the next time point (pink) the OSS guidewire 30 tip is beyond the over-the-wire tip and a new curve (2) has appeared. In this manner, the curves can be calculated and a threshold defined for when the OSS guidewire 30 exits the catheter 40.

Referring back to FIG. 9, a stage S524 of flowchart 520 encompasses an identification of an exit point of OSS guidewire 30 from catheter 40 whereby the exit point defines a length of the catheter 40 during a stage S526 of flowchart 520.

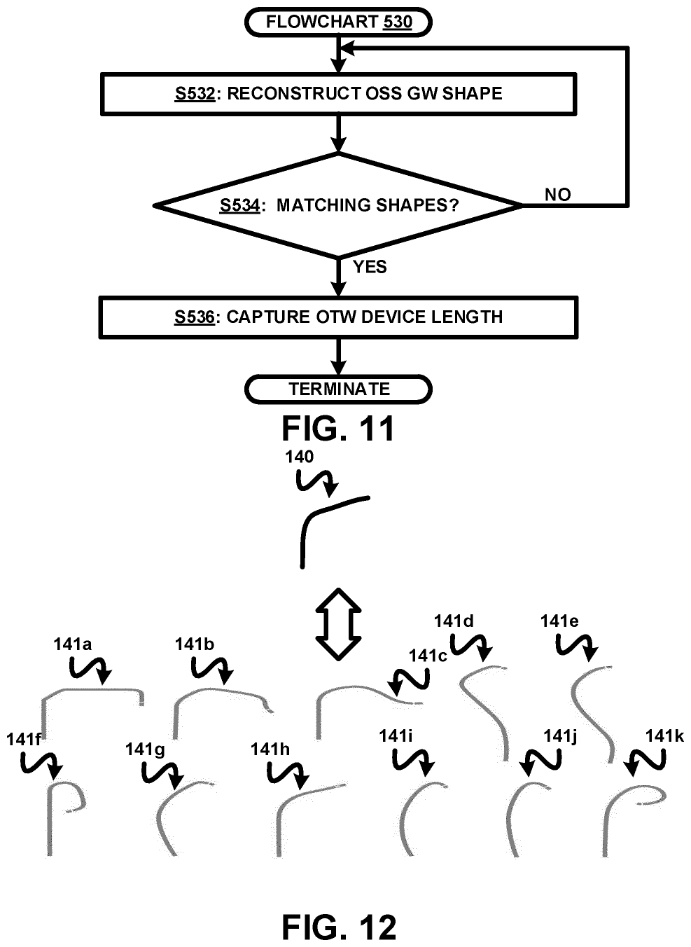

FIG. 11 illustrates a flowchart 530 represents a OSS distal curvature method of the present disclosure. Referring to FIG. 11, a stage S532 of flowchart 530 encompasses controller 20 receiving a current reconstructed shape of OSS guidewire 30, and a stage S534 of flowchart 530 encompasses measurer 22 attempting match the reconstruction of OSS guidewire 30 to pre-defined curves of a catheter as stored in a look-up table of an array of the pre-defined curves and associated lengths. Stage S532 and 534 are executed in a loop until controller 20 identifies a match with a lowest error, which indicates a shape of OSS guidewire 30 that most resembles the tips of OSS guidewire 30 and catheter 40 being aligned. A stage S536 of flowchart 530 encompassed controller 20 capturing the length of the match pre-defined catheter as the length of catheter 40.

FIG. 12 illustrates a current reconstructed shape 140 of OSS guidewire 50 being matched to a multipurpose A2 catheter 141h among a look-up table of a tiger catheter 141a, a jacky catheter 141b, an amplatz left catheter 141c, a LCB catheter 141d, a RCB catheter 141e, a Judkins left catheter 141f, a Judkins right catheter 141g, a multipurpose A2 catheter 141h, a IM catheter 141i, a 3D lima catheter 141j and an IM VB-1 catheter 141k.

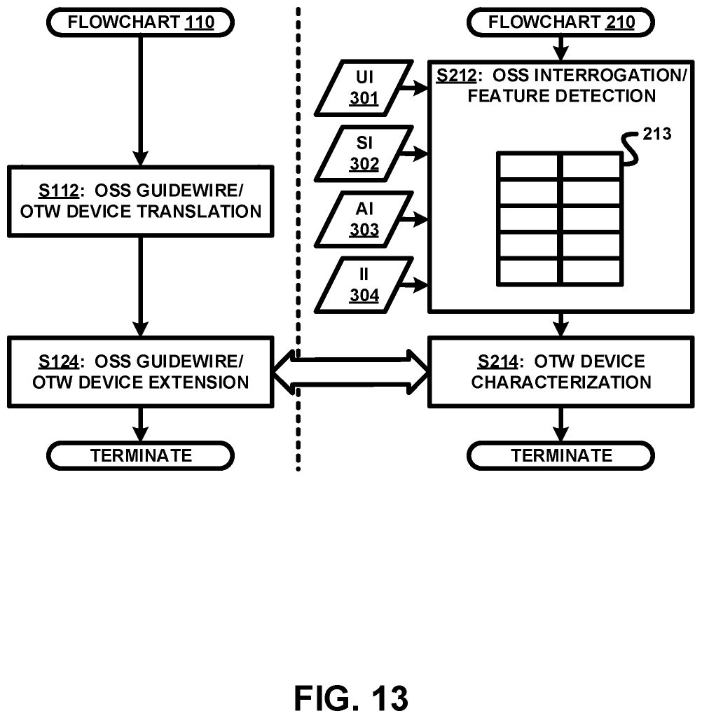

FIG. 13 illustrates flowcharts 110 and 210 representative of an OSS registration method of the present disclosure that is particularly applicable to an OTW device without an attached hub, such as, for example, catheter 41 of FIG. 1.

Referring to FIG. 13, flowchart 110 and flowchart 210 encompass a workflow of user actions and controller executions for determining registration characteristics defining a spatial tracking of catheter 41 or another OTW device without an attached hub.

A stage S112 of flowchart 110 encompasses a first user action of manually or robotically translating OSS guidewire 30 through catheter 41, and stage S114 of flowchart 110 encompasses a second user action of manually or robotically extending a tip of OSS guidewire 30 from a distal tip of catheter 41. Stages S112 and S114 of flowchart 110 initiate stage S212 and stage S214 of flowchart 210 for an automatic measurement of a length of catheter 41.

The initiation of stage S212 and S214 of flowchart 210 occur from one or more of the following inputs.

In one exemplary embodiment, a user input 301 may provide an indication to controller 20 that an insertion of OSS guidewire 30 into catheter 41 is about to start. User input 310 may be in the form of visual, verbal and/or manual cues.

In a second exemplary embodiment, a sensor may incorporated into the catheter 41 whereby the sensor sends a sensor input signal 302 to controller 20 when the sensor senses OSS guidewire 30 is in contact with the catheter 41. Further, additional sensors may be incorporated into other devices, at the table, in the room, etc. for sensing stage S112 and/or S114 have occurred.

In a third exemplary embodiment, controller 20 (or another controller) may execute an algorithm continuously looking at the shape data from the OSS guidewire 30 to detect a formation of a minimal curvature in a distal end of OSS guidewire 30. Once the algorithm sees the minimal curvature in the shape data, an algorithm signal 303 triggers stage S212.

In a fourth exemplary embodiment, imaging information 304 extracted from x-ray, ultrasound, optical/camera, etc. imaging may also be used to identify when OSS guidewire 30 is proximate or in contact with the catheter 41. The identification is used as a trigger to initiate stage S212.

Stages S212 and S214 involve a continuous processing of the shape sensing data of OSS guidewire 30 during stage S212 as the OSS guidewire is translated through catheter 41 during stage S102 whereby, during stage S214, controller 20 attempts to match the current shape data of OSS guidewire 30 to the pre-defined catheter shapes in a look-up table (LUT) 213. The pre-defined catheter shape with the lowest error is defined as the catheter type with the best match and that catheter type (or classification) is saved whereby registration characteristics may be derived therefrom.

In one embodiment of stage S214, controller 20 receiving a current reconstructed shape of OSS guidewire 30 and attempts to match the reconstruction of OSS guidewire 30 to pre-defined curves of a catheter as stored in a look-up table of an array of the pre-defined curves and associated characteristics including type of hub. Controller 20 identifies a match with a lowest error, which indicates a shape of OSS guidewire 30 that most resembles the tips of OSS guidewire 30 and catheter 41 being aligned, and controller 20 capture the registration characteristics of the matched pre-defined catheter shape as the registration characteristics of catheter 41.

As previously described, FIG. 12 illustrates a current reconstructed shape 140 of OSS guidewire 50 being matched to a multipurpose A2 catheter 141h among a look-up table of a tiger catheter 141a, a jacky catheter 141b, an amplatz left catheter 141c, a LCB catheter 141d, a RCB catheter 141e, a Judkins left catheter 141f, a Judkins right catheter 141g, a multipurpose A2 catheter 141h, a IM catheter 141i, a 3D lima catheter 141j and an IM VB-1 catheter 141k. As such, the registration characteristics of multipurpose A2 catheter 141h become the registration characteristics of catheter 41, particularly the type of hub to be attached to catheter 41.

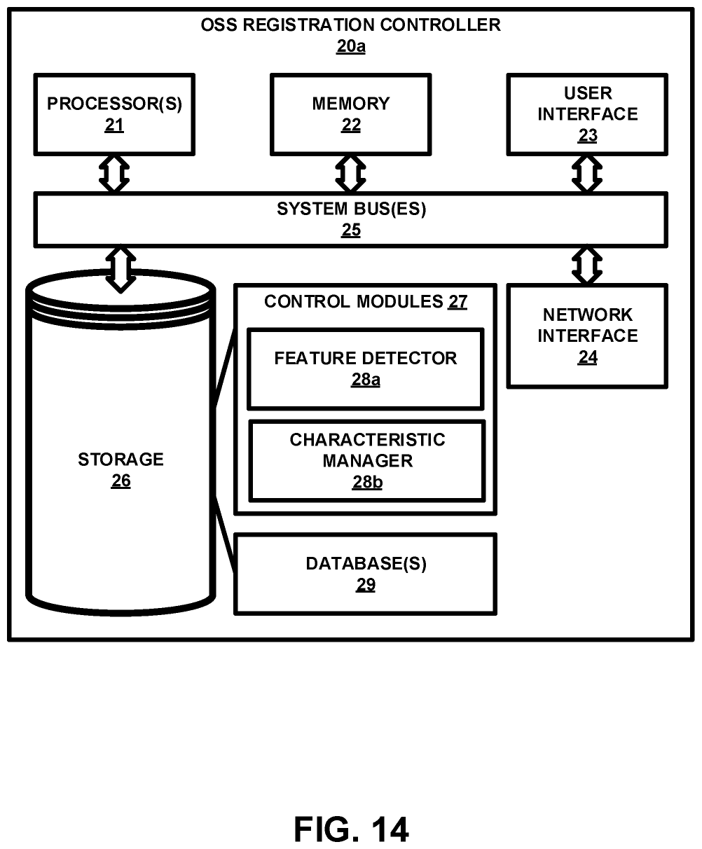

To facilitate a further understanding of the present disclosure, the following description of FIG. 14 teaches exemplary embodiments of an OSS registration controller in accordance with the present disclosure. From the description of FIG. 14, those having ordinary skill in the art of the present disclosure will appreciate how to apply the present disclosure to make and use additional embodiments of an OSS registration controller in accordance with the present disclosure.

Referring to FIG. 14, an exemplary embodiment 20a of OSS registration controller 20 (FIG. 1) includes one or more processor(s) 21, memory 22, a user interface 23, a network interface 24, and a storage 26 interconnected via one or more system buses 25.

Each processor 21 may be any hardware device, as known in the art of the present disclosure or hereinafter conceived, capable of executing instructions stored in memory 22 or storage or otherwise processing data. In a non-limiting example, the processor(s) 21 may include a microprocessor, field programmable gate array (FPGA), application-specific integrated circuit (ASIC), or other similar devices.

The memory 22 may include various memories, as known in the art of the present disclosure or hereinafter conceived, including, but not limited to, L1, L2, or L3 cache or system memory. In a non-limiting example, the memory 22 may include static random access memory (SRAM), dynamic RAM (DRAM), flash memory, read only memory (ROM), or other similar memory devices.

The user interface 23 may include one or more devices, as known in the art of the present disclosure or hereinafter conceived, for enabling communication with a user such as an administrator. In a non-limiting example, the user interface may include a command line interface or graphical user interface that may be presented to a remote terminal via the network interface 24.

The network interface 24 may include one or more devices, as known in the art of the present disclosure or hereinafter conceived, for enabling communication with imaging systems (not shown) and additional tracking systems (not shown) (e.g., an electromagnetic tracking system. In a non-limiting example, the network interface 24 may include a network interface card (NIC) configured to communicate according to the Ethernet protocol. Additionally, the network interface 26 may implement a TCP/IP stack for communication according to the TCP/IP protocols. Various alternative or additional hardware or configurations for the network interface 26 will be apparent.

The storage 26 may include one or more machine-readable storage media, as known in the art of the present disclosure or hereinafter conceived, including, but not limited to, read-only memory (ROM), random-access memory (RAM), magnetic disk storage media, optical storage media, flash-memory devices, or similar storage media. In various non-limiting embodiments, the storage 26 may store instructions for execution by the processor(s) 21 or data upon with the processor(s) 21 may operate. For example, the storage 26 may store a base operating system for controlling various basic operations of the hardware. The storage 26 also stores a feature detector 28a and a characteristic manager 28b as application modules in the form of executable software/firmware for implementing the various functions of feature detection and registration characteristic determination as previously described in the present disclosure. The storage 26 may also store a OSS interrogation application module (not shown) and/or a OSS reconstruction application module (not shown) as known in the art of the present disclosure or hereinafter conceived. Furthermore, storage 26 may store look-up table as previously described in the present disclosure (e.g., pre-defined OTW shapes).

In practice, OSS registration controller 20a may be incorporated within a stand-alone workstation (e.g., a desktop, a laptop, a pad or a smart phone) or may be incorporated within a workstation or a server of an OSS system.

Referring to FIGS. 1-14, those having ordinary skill in the art of the present disclosure will appreciate numerous benefits of the present disclosure including, but not limited to, for automatically detecting features of an OSS guidewire to thereby automatically determine the required registration characteristics of an OTW device needed for an accurate, robust spatial tracking of the OTW device by OSS guidewire or other tracking device that is applicable to numerous and various applications. The device characteristics that are determined may be used for the following (but not limited to): recalling visualization properties, annotating the procedure, logging important information, documentation, or setting device/imaging properties for improving procedural accuracy.

Furthermore, exemplary embodiments described in the present disclosure teach how a feature of an OSS guidewire may be detected to thereby define a device characteristics of a OTW device (e.g., a length of a catheter). Within the same principles of the present disclosure, a feature of an OSS guidewire may be detected to thereby delineate an anatomical location. For example, a specific shape of the OSS guidewire (or OSS guidewire and OTW device) is caused by an anatomical location whereby the detected shape of the OSS guidewire (and OTW device) and anatomical location may be stored in a pre-defined LUT. As a result, during a subsequent procedure, a detection of the feature facilitates a determination of the anatomical location or a particular type of procedure via the LUT.

Further, as one having ordinary skill in the art will appreciate in view of the teachings provided herein, structures, elements, components, etc. described in the present disclosure/specification and/or depicted in the Figures may be implemented in various combinations of hardware and software, and provide functions which may be combined in a single element or multiple elements. For example, the functions of the various structures, elements, components, etc. shown/illustrated/depicted in the Figures can be provided through the use of dedicated hardware as well as hardware capable of executing software in association with appropriate software for added functionality. When provided by a processor, the functions can be provided by a single dedicated processor, by a single shared processor, or by a plurality of individual processors, some of which can be shared and/or multiplexed. Moreover, explicit use of the term “processor” or “controller” should not be construed to refer exclusively to hardware capable of executing software, and can implicitly include, without limitation, digital signal processor (“DSP”) hardware, memory (e.g., read only memory (“ROM”) for storing software, random access memory (“RAM”), non-volatile storage, etc.) and virtually any means and/or machine (including hardware, software, firmware, combinations thereof, etc.) which is capable of (and/or configurable) to perform and/or control a process.

Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future (e.g., any elements developed that can perform the same or substantially similar function, regardless of structure). Thus, for example, it will be appreciated by one having ordinary skill in the art in view of the teachings provided herein that any block diagrams presented herein can represent conceptual views of illustrative system components and/or circuitry embodying the principles of the invention. Similarly, one having ordinary skill in the art should appreciate in view of the teachings provided herein that any flow charts, flow diagrams and the like can represent various processes which can be substantially represented in computer readable storage media and so executed by a computer, processor or other device with processing capabilities, whether or not such computer or processor is explicitly shown.

Having described preferred and exemplary embodiments of the various and numerous inventions of the present disclosure (which embodiments are intended to be illustrative and not limiting), it is noted that modifications and variations can be made by persons skilled in the art in light of the teachings provided herein, including the Figures. It is therefore to be understood that changes can be made in/to the preferred and exemplary embodiments of the present disclosure which are within the scope of the embodiments disclosed herein.

Moreover, it is contemplated that corresponding and/or related systems incorporating and/or implementing the device/system or such as may be used/implemented in/with a device in accordance with the present disclosure are also contemplated and considered to be within the scope of the present disclosure. Further, corresponding and/or related method for manufacturing and/or using a device and/or system in accordance with the present disclosure are also contemplated and considered to be within the scope of the present disclosure.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measured cannot be used to advantage. Any reference signs in the claims should not be construed as limiting the scope.

1. An optical shape sensing registration system, comprising:
an optical shape sensing guidewire translatable within an over-the-wire device; and
an optical shape sensing registration controller for controlling an autonomous device registration of the over-the-wire device, wherein the optical shape sensing registration controller is configured to at least one of:
automatically detect at least one sensing feature of the optical shape sensing guidewire from an optical shape sensing of a translation of the optical shape sensing guidewire within the over-the-wire device; and
automatically determine at least one registration characteristic of the over-the-wire device from an automatic detection of the at least one sensing feature of the optical shape sensing guidewire.

2. The optical shape sensing registration system of claim 1,
wherein the at least one registration characteristic of the over-the-wire device is a hub; and
wherein the optical shape sensing registration controller is configured to automatically identify the hub from the automatic detection of the at least one sensing feature of the optical shape sensing guidewire.

3. The optical shape sensing registration system of claim 2,
wherein the hub includes a hub template; and
wherein, when the hub is attached to the over-the-wire device, the optical shape sensing registration controller being configured to automatically identify the hub from the automatic detection of the at least one sensing feature of the optical shape sensing guidewire includes the optical shape sensing registration controller configured to:
compare the optical shape sensing of a translation of the optical shape sensing guidewire through the hub to a plurality of pre-defined hub templates; and
select a one of the pre-defined hub templates having a best shape match to the hub template.

4. The optical shape sensing registration system of claim 2,
wherein, when the hub is unattached to the over-the-wire device, the optical shape sensing registration controller being configured to automatically identify the hub from the automatic detection of the at least one sensing feature of the optical shape sensing guidewire includes the optical shape sensing registration controller configured to:
compare the optical shape sensing of the translation of the optical shape sensing guidewire through the over-the-wire device to a shape of each of a plurality of pre-defined over-the-wire devices;
select a one of the pre-defined over-the-wire devices having a best shape match to the optical shape sensing of the translation of the optical shape sensing guidewire through the over-the-wire device; and
identify the hub associated with the selected one of the pre-defined over-the-wire devices.

5. The optical shape sensing registration system of claim 1, wherein the optical shape sensing registration controller being configured to automatically determine the at least one registration characteristic of the over-the-wire device from an automatic detection of the at least one sensing feature of the optical shape sensing guidewire includes the optical shape sensing registration controller being configured to:
measure a length of the over-the-wire device based on an analysis of a curvature of the optical shape sensing of the translation of the optical shape sensing guidewire through the over-the-wire device.

6. The optical shape sensing registration system of claim 5, wherein the optical shape sensing registration controller being configured to measure the length of the over-the-wire device includes the optical shape sensing registration controller being configured to:
plot curvatures of the optical shape sensing guidewire relative to positions of a reference along the optical shape sensing guidewire; and
measure the length of the over-the-wire device relative to the reference from a position of the reference along the optical shape sensing guidewire corresponding to a highest peak of the curvatures.

7. The optical shape sensing registration system of claim 5, wherein the optical shape sensing registration controller being configured to measure the length of the over-the-wire device includes the optical shape sensing registration controller being configured to:
plot curvatures of the optical shape sensing guidewire relative to different positions of the optical shape sensing guidewire within the over-the-wire device; and
measure the length of the over-the-wire device relative to a reference from a position of the optical shape sensing guidewire within the over-the-wire device corresponding to the curvature of the optical shape sensing guidewire indicative of a distal tip of the over-the-wire device.

8. The optical shape sensing registration system of claim 5, wherein the optical shape sensing registration controller being configured to measure the length of the over-the-wire device includes the optical shape sensing registration controller being configured to:
match the optical shape sensing of the translation of the optical shape sensing guidewire through the over-the-wire device to a plurality of pre-defined over-the-wire device shapes; and
measure the length of the over-the-wire device relative to a reference from a one of the plurality of pre-defined over-the-wire device shapes of the over-the-wire devices having a best shape match to the optical shape sensing of the translation of the optical shape sensing guidewire through the over-the-wire device.

9. An optical shape sensing registration controller for controlling an autonomous device registration the over-the-wire device, the optical shape sensing registration controller comprising:
a non-transitory machine-readable storage medium encoded with instructions for execution by at least one processor, the non-transitory machine-readable storage medium including the instructions to:
automatically detect at least one sensing feature of the optical shape sensing guidewire from an optical shape sensing of a translation of the optical shape sensing guidewire within the over-the-wire device; and
automatically determine at least one registration characteristic of the over-the-wire device from an automatic detection of the at least one sensing feature of the optical shape sensing guidewire.

10. The optical shape sensing registration controller of claim 9,
wherein the at least one registration characteristic of the over-the-wire device is a hub; and
wherein the instructions to automatically determine the at least one registration characteristic of the over-the-wire device from the automatic detection of the at least one sensing feature of the optical shape sensing guidewire includes instructions to:
automatically identify the hub from the automatic detection of the at least one sensing feature of the optical shape sensing guidewire.

11. The optical shape sensing registration controller of claim 10,
wherein the hub includes a hub template; and
wherein, when the hub is attached to the over-the-wire device, the instructions to automatically identify the hub from the automatic detection of the at least one sensing feature of the optical shape sensing guidewire includes instructions to:
compare the optical shape sensing of a translation of the optical shape sensing guidewire through the hub to a plurality of pre-defined hub templates; and
select a one of the pre-defined hub templates having a best shape match to the hub template.

12. The optical shape sensing registration controller of claim 10, wherein, when the hub is unattached to the over-the-wire device, the instructions to automatically identify the hub from the automatic detection of the at least one sensing feature of the optical shape sensing guidewire includes instructions to:
compare the optical shape sensing of the translation of the optical shape sensing guidewire through the over-the-wire device to a shape of each of a plurality of pre-defined over-the-wire devices;
select a one of the pre-defined over-the-wire devices having a best shape match to the optical shape sensing of the translation of the optical shape sensing guidewire through the over-the-wire device; and
identify the hub associated with the selected one of the pre-defined over-the-wire devices.

13. The optical shape sensing registration controller of claim 10, wherein the optical shape sensing registration controller being configured to automatically determine the at least one registration characteristic of the over-the-wire device from an automatic detection of the at least one sensing feature of the optical shape sensing guidewire include instructions to:
plot curvatures of the optical shape sensing guidewire relative to positions of a reference along the optical shape sensing guidewire; and
measure a length of the over-the-wire device relative to the reference from a position of the reference along the optical shape sensing guidewire corresponding to a highest peak of the curvatures.

14. The optical shape sensing registration controller of claim 10, wherein the optical shape sensing registration controller being configured to automatically determine the at least one registration characteristic of the over-the-wire device from an automatic detection of the at least one sensing feature of the optical shape sensing guidewire include instructions to:
plot curvatures of the optical shape sensing guidewire relative to different positions of the optical shape sensing guidewire within the over-the-wire device; and
measure a length of the over-the-wire device relative to a reference from a position of the optical shape sensing guidewire within the over-the-wire device corresponding to the curvature of the optical shape sensing guidewire indicative of a distal tip of the over-the-wire device.

15. The optical shape sensing registration controller of claim 10, wherein the optical shape sensing registration controller being configured to automatically determine the at least one registration characteristic of the over-the-wire device from an automatic detection of the at least one sensing feature of the optical shape sensing guidewire include instructions to:
match the optical shape sensing of the translation of the optical shape sensing guidewire through the over-the-wire device to a plurality of pre-defined over-the-wire device shapes; and
measure a length of the over-the-wire device relative to a reference from a one of the plurality of pre-defined over-the-wire device shapes of the over-the-wire devices having a best shape match to the optical shape sensing of the translation of the optical shape sensing guidewire through the over-the-wire device.

16. An optical shape sensing registration method executable by an optical shape sensing registration controller for controlling an autonomous device registration of an over-the-wire device, the optical shape sensing registration method comprising:
controlling a translation of an optical shape sensing guidewire within the over-the-wire device; and
automatically detecting, via the optical shape sensing registration controller, at least one sensing feature of the optical shape sensing guidewire from an optical shape sensing of a translation of the optical shape sensing guidewire within the over-the-wire device; and
automatically determining, via the optical shape sensing registration controller, at least one registration characteristic of the over-the-wire device from the automatic detection of the at least one sensing feature of the optical shape sensing guidewire.

17. The optical shape sensing registration method of claim 16,
wherein the at least one registration characteristic of the over-the-wire device is a hub; and
wherein the automatically determining, via the optical shape sensing registration controller, the at least one registration characteristic of the over-the-wire device from the automatic detection of the at least one sensing feature of the optical shape sensing guidewire includes:
automatically identifying, via the optical shape sensing registration controller, the hub from the automatic detection of the at least one sensing feature of the optical shape sensing guidewire.

18. The optical shape sensing registration method of claim 17,
wherein the hub includes a hub template; and
wherein, when the hub is attached to the over-the-wire device, the automatically determining, via the optical shape sensing registration controller, the at least one registration characteristic of the over-the-wire device from the automatic detection of the at least one sensing feature of the optical shape sensing guidewire includes:
comparing, via the optical shape sensing registration controller, the optical shape sensing of a translation of the optical shape sensing guidewire through the hub to a plurality of pre-defined hub templates; and
selecting, via the optical shape sensing registration controller, a one of the pre-defined hub templates having a best shape match to the hub template.

19. The optical shape sensing registration method of claim 17,
wherein, when the hub is unattached to the over-the-wire device, the automatically determining, via the optical shape sensing registration controller, the at least one registration characteristic of the over-the-wire device from the automatic detection of the at least one sensing feature of the optical shape sensing guidewire includes:
comparing, via the optical shape sensing registration controller, the optical shape sensing of the translation of the optical shape sensing guidewire through the over-the-wire device to a shape of each of a plurality of pre-defined over-the-wire devices;
selecting, via the optical shape sensing registration controller, a one of the pre-defined over-the-wire devices having a best shape match to the optical shape sensing of the translation of the optical shape sensing guidewire through the over-the-wire device; and
identifying, via the optical shape sensing registration controller, the hub associated with the selected one of the pre-defined over-the-wire devices.

20. The optical shape sensing registration method of claim 17, wherein the automatically determining, via the optical shape sensing registration controller, the at least one registration characteristic of the over-the-wire device from the automatic detection of the at least one sensing feature of the optical shape sensing guidewire includes:
measuring, via the optical shape sensing registration controller, a length of the over-the-wire device based on an analysis of a curvature of the optical shape sensing of the translation of the optical shape sensing guidewire through the over-the-wire device.
